(19) United States
(12) Patent Application Publication
(10) Pub. No.: US 2007/0055228 A1
(43) Pub. Date: Mar. 8, 2007

(54) Ultrasonic scalpel device

(76) Inventors: Howard K. Berg, Owings Mills, MD (US); Justin Somerville, Owings Mills, MD (US)

Correspondence Address: ROBERTS, MLOTKOWSKI & HOBBES, P. O. BOX 10064, MCLEAN, VA, 22102-8064, US

(21) Appl. No.: 11/491,195

(22) Filed: Jul. 24, 2006

Related U.S. Application Data
(60) Provisional application No. 60/749,614, filed on Dec. 13, 2005.
(60) Provisional application No. 60/701,503, filed on Jul. 22, 2005.

(51) Int. Cl. A61B 18/18

(52) U.S. Cl. 606/41

(57) ABSTRACT

The present invention relates, in general, to ultrasonic surgical instruments and, more particularly, to an ultrasonic surgical clamp coagulator apparatus (or ultrasonic scalpel) particularly configured to provide hand activation configured in such a way to provide an ergonomic grip and improved control of the scalpel and ease in controlling or activating applied energy during surgical operation.

This application claims the benefit under 35 U.S.C. §119(e) of U.S. Provisional Application No. 60/749,614, filed Dec. 13, 2005, and U.S. Provisional Application No. 60/701,503, filed Jul. 22, 2005, the entire contents of both of which are incorporated by reference.

### BACKGROUND OF THE INVENTION

1. Field of the Invention

The present invention relates, in general, to ultrasonic surgical instruments and, more particularly, to an ultrasonic surgical clamp coagulator apparatus (or ultrasonic scalpel) particularly configured to provide hand activation configured in such a way to provide an ergonomic grip and improved control of the scalpel and ease in controlling or activating applied energy during surgical operation.

2. Description of the Related Art

Ultrasonic surgical instruments are finding increasingly widespread applications in surgical procedures by virtue of the unique performance characteristics of such instruments. Depending upon specific instrument configurations and operational parameters, ultrasonic surgical instruments can provide substantially simultaneous cutting of tissue and hemostasis by coagulation, desirably minimizing patient trauma. The cutting action is typically effected by an end-effector at the distal end of the instrument, which transmits ultrasonic energy to tissue brought into contact with the end-effector. Ultrasonic instruments of this nature can be configured for open surgical use, laparoscopic or endoscopic surgical procedures including robotic-assisted procedures. An example of an ultrasonic scalpel is the Harmonic Scalpel available from Ethicon Endo-Surgery (Cincinnati, Ohio), with one particular instrument available under the name Harmonic ACE.

Ultrasonic surgical instruments have been developed that include a clamp member to press tissue against the blade of the end-effector in order to couple ultrasonic energy to the tissue of a patient. Such an arrangement (sometimes referred to as a clamp coagulator shears or an ultrasonic transector) is disclosed in U.S. Pat. Nos. 5,322,055; 5,873,873 and 6,325,811, all of which are incorporated herein by reference. The surgeon activates the clamp arm to press the clamp pad against the blade by squeezing on the handgrip or handle. Other current clamp coagulator designs utilizing a pistol grip are known in the art. For example, WO 2006/042210, which is incorporated herein by reference, discloses a clamp coagulator having a pistol grip design.

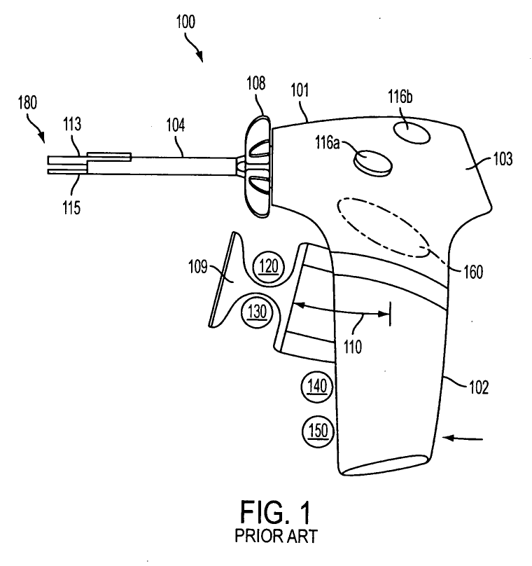

One current design of an ultrasonic scalpel surgical system 100 having a pistol grip configuration is depicted in FIG. 1. The ultrasonic scalpel handle assembly 101 includes a body portion 103 and a handle portion 102, the handle portion 102 intersecting the body portion 103 at an angle of about 90°. The ultrasonic scalpel surgical system 100 includes an end-effector 180 comprising a blade 115 and a clamp member 113 mounted on a shaft 104 extending from the handle body 101. Two separate activation buttons 116a and 116b are located on the body portion 103 of the handle body 101 for controlling the level of applied ultrasound energy at two levels (e.g., cut, and coagulate). As illustrated, the activation buttons 116a and 116b are placed in the range of the natural swing of the surgeon's thumb, whether gripping surgical instrument right-handed or left handed. A trigger 109 for opening and closing the clamp member 113 against the blade 115 is located on the front of the handle portion 102 of the handle body 101 while it is moved along its range of motion 110. During operation, the trigger 109 is typically gripped by the surgeon's index finger 120 and middle finger 130, while the handle portion 102 is gripped by the surgeon's ring finger 140 and little finger 150.

It would be desirable to provide an ultrasonic surgical instrument that overcomes some of the deficiencies of current instruments.

### SUMMARY OF THE INVENTION

The scalpel according to the present invention overcomes one or more of the problems with existing scalpels. The scalpel according to the present invention is particularly configured to provide hand activation configured in such a way to provide an ergonomic grip and improved control of the scalpel and ease in controlling or activating applied energy during surgical operation.

An embodiment of the present invention relates to a directed energy surgical instrument. The directed energy may be ultrasonic energy, or an electrocautery energy. The directed energy surgical instrument comprises a handle assembly which includes a body portion and a handle portion, the body portion and handle portion arranged in a pistol-shaped configuration. An elongate transmission assembly is attached to the body portion of the handle assembly and extends distally therefrom. The transmission assembly comprises a tubular sheath member attached at its proximal end to the body portion. An actuating member is disposed within the tubular sheath member. An end-effector assembly attached to the actuating member adjacent the distal end of the tubular sheath member, the end effector assembly comprising a blade member and a clamp member that is pivotable relative to the blade member. The actuating member is configured for transmitting energy from the handle assembly to the end effector assembly. A trigger is mounted to the handle portion. The trigger is configured so that a portion of the trigger extends distally from a distal surface of the handle portion and is operatively connected to the actuating member so that motion of the trigger causes the clamp member to pivot relative to the blade member. An activation mechanism or activation means is adapted to control the transmission of energy to the end effector assembly. The activation mechanism or activation means is located on the distal surface of the handle portion intermediate the trigger and the body portion.

In one embodiment, the handle portion and body portion of the handle assembly define an angle, the angle being greater than about 100° and less than about 120°, such as greater than 100° and less than 120°, e.g., about 110°, or 110°. In one embodiment, the handle assembly can be gripped such that the index finger and middle finger contact the activation mechanism while the ring finger and little finger contact the trigger.

### DETAILED DESCRIPTION OF THE DRAWINGS

The features of the invention are set forth with particularity in the appended claims. The invention itself, however, both as to organization and methods of operation, may best be understood by reference to the following description, taken in conjunction with the accompanying drawings in which:

FIG. 1 depicts a conventional ultrasonic scalpel wherein the body has a pistol-shaped design;

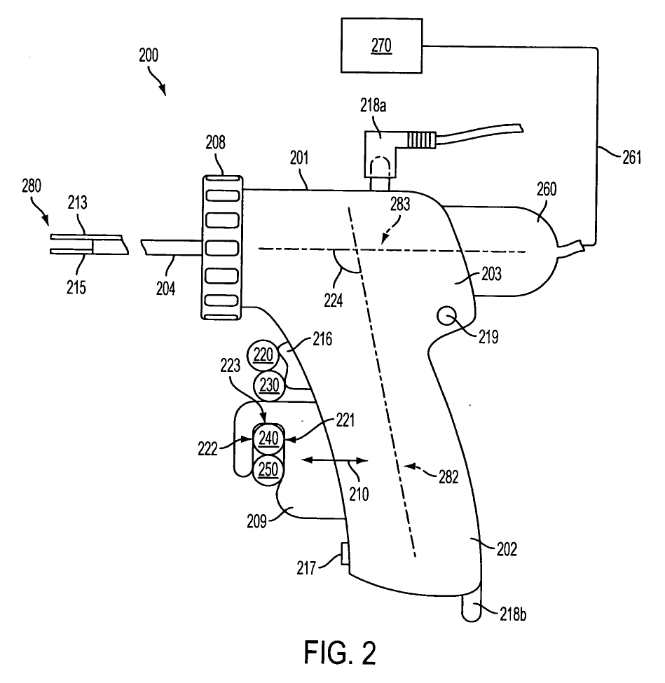

FIG. 2 depicts an ultrasonic scalpel according to an embodiment of the invention wherein the body has a pistol-shaped design;

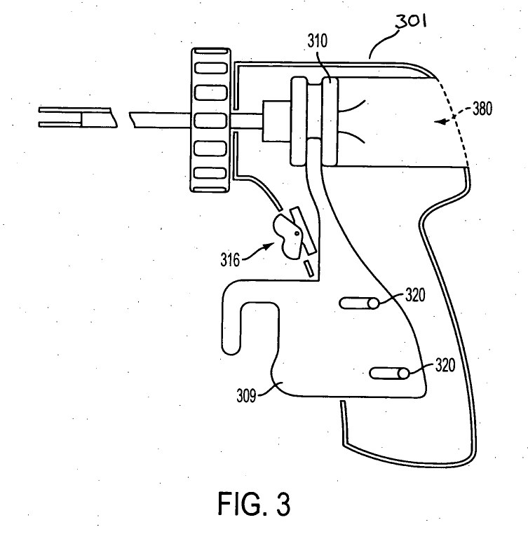

FIG. 3 depicts a cut away view of the ultrasonic scalpel according to an embodiment of the invention wherein the body has a pistol-shaped design;

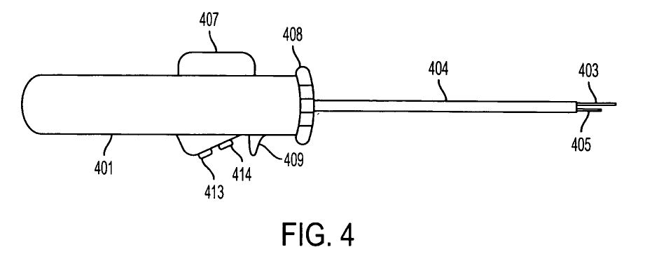

FIG. 4 depicts an ultrasonic scalpel according to an embodiment of the invention wherein the body has a barrel-shaped design;

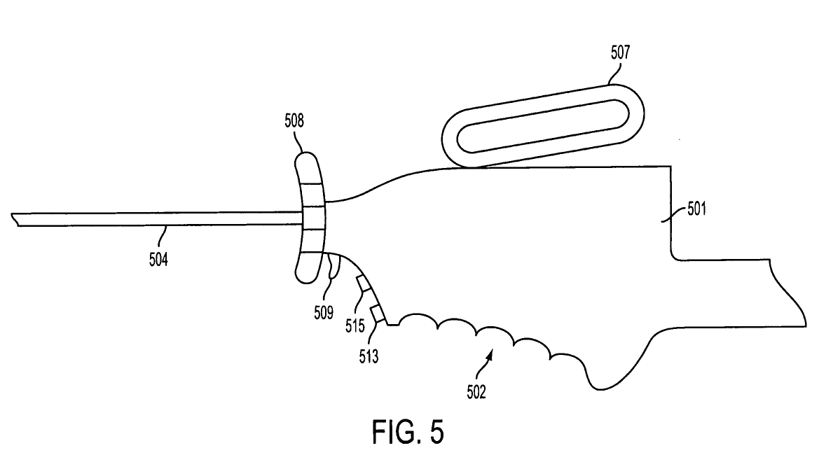

FIG. 5 depicts an ultrasonic scalpel according to an embodiment of the invention wherein the body has an inclined grip portion and a blade closure handle opposite the grip portion;

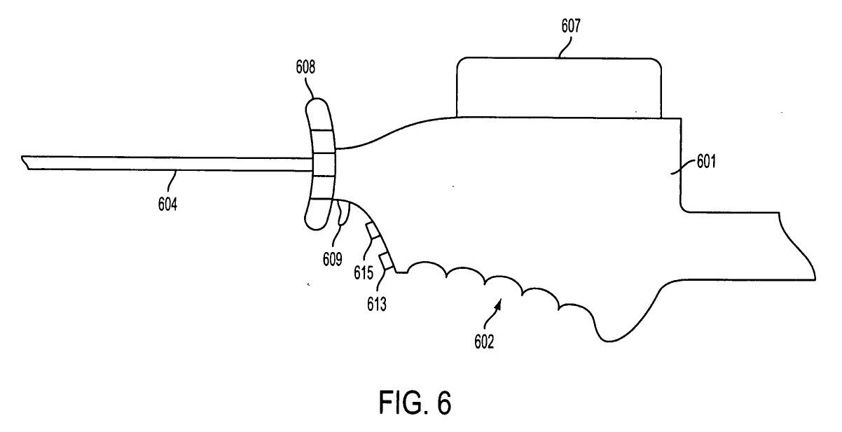

FIG. 6 depicts an ultrasonic scalpel according to an embodiment of the invention wherein the body has an inclined grip portion and a blade closure button opposite the grip portion;

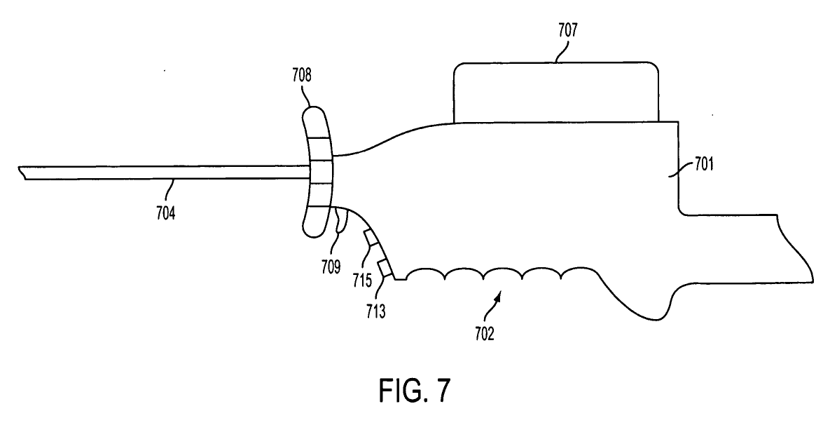

FIG. 7 depicts an ultrasonic scalpel according to an embodiment of the invention wherein the body has a grip portion and a blade closure button opposite the grip portion.

### DETAILED DESCRIPTION OF THE PREFERRED EMBODIMENTS

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Further, unless otherwise indicated, the terms and expressions employed herein have been chosen for the purpose of describing the illustrative embodiments of the present invention for the convenience of the reader and are not for the purpose of limiting the invention. Further, it is understood that any one or more of the following-described embodiments, expressions of embodiments, and examples, can be combined with any one or more of the other following-described embodiments, expressions of embodiments, and examples.

The present invention is particularly directed to an improved directed energy surgical instrument, e.g., a surgical clamp coagulator apparatus, ultrasonic scalpel, an electrocautery apparatus or similar device, having an ergonomic grip with improved control of the instrument and applied energy during surgical operation. The present apparatus can be readily configured for use in open surgical procedures, as well as laparoscopic or endoscopic procedures and robot-assisted surgical procedures. Versatile use is facilitated by selective use of directed energy, which may include ultrasonic or electrocautery energy. When ultrasonic components of the apparatus are inactive, tissue can be readily gripped and manipulated, as desired, without tissue cutting or damage. When the ultrasonic components are activated, the apparatus permits tissue to be gripped for coupling with the ultrasonic energy to effect tissue coagulation, with application of increased pressure efficiently effecting tissue cutting and coagulation. If desired, ultrasonic energy can be applied to tissue without use of the clamp arm of the apparatus by appropriate manipulation of the ultrasonic blade. Alternatively, the present invention may utilize electrocautery energy instead of or in addition to ultrasound.

As will become apparent from the following description, the present surgical clamp coagulator apparatus is particularly configured for disposable use by virtue of its straightforward construction. As such, it is contemplated that the apparatus be used in association with an ultrasonic generator unit of a surgical system, whereby ultrasonic energy from the generator unit provides the desired ultrasonic actuation for the present clamp coagulator apparatus. It will be appreciated that a clamp coagulator apparatus embodying the principles of the present invention can be configured for non-disposable or multiple use, and non-detachably integrated with an associated ultrasonic generator unit. However, detachable connection of the present clamp coagulator apparatus with an associated ultrasonic generator unit is presently preferred for single-patient use of the apparatus.

The present invention will be described as applied to a particular surgical instrument as described herein. However, such description is exemplary only, and is not intended to limit the scope and applications of the invention. For example, the invention is useful in combination with a multitude of ultrasonic instruments including those described in, for example, U.S. Pat. Nos. 5,938,633; 5,935,144; 5,944,737; 5,322,055, 5,630,420; 5,449,370; or WO 2006/042210.

Applicants have found that when the activation buttons 116a, 116b are placed in the natural swing of the surgeon's thumb (i.e. near the top of the body portion of the handle assembly) in scalpels such as that depicted in FIG. 1, surgeons encounter certain difficulties in operating the scalpel during surgical procedures. Specifically actuation of buttons 116a, 116b with the thumb can be uncomfortable, which can lead to hand fatigue. Applicants have also found that in certain positions, activating the device requires the use of two hands—one hand on the trigger and another on the buttons 116a, 116b. Additionally, the use of two separate buttons for controlling high and low levels of applied energy to the scalpel tip can lead to error in selecting the correct level of applied energy during surgery and/or increase the amount of time required to complete a given procedure. These and other problems can be exacerbated when the handle is rotated into less familiar positions during surgery, making it more difficult to locate, distinguish between, and/or operate the different buttons.

Applicants have further found that in scalpels where the axis of the body portion intersects the axis of the handle portion at a roughly 90° angle, e.g., as that of FIG. 1, the scalpel must be held in a wrist-bent position for prolonged periods during certain surgical procedures. This places stress on the wrist as it is held in a stiff, unnatural position for extended time periods, which can further lead to wrist fatigue and related conditions.

Often the surgeon must twist and/or turn his body to avoid losing control of the scalpel when performing surgical procedures with a scalpel having one or more of the above shortcomings. This can lead to injury and/or fatigue and reduces the pool of surgeons who can use the device. Some surgeons who have used scalpels with one or more of the above problems have complained of developing tennis elbow, low back pain, sciatica, and other muscular and joint problems.

In one embodiment of our invention, a handle assembly of a pistol-shaped scalpel is provided comprising a handle portion and a body portion, where a trigger is provided on the handle portion, and activation mechanism is provided on the handle portion intermediate the trigger and the body portion. This arrangement permits lowering the trigger to a more normal hand/grip position. This arrangement also provides an advantage in that it permits the surgeon to operate the trigger using the ring and little fingers, which have increased grip strength relative to the index and middle fingers. In addition, this arrangement frees the index and middle finger, which are capable of fine movement, to manipulate the activation mechanism during operation.

In one embodiment, a handle assembly of a pistol-shaped scalpel is provided where the axis of the handle portion and the axis of the body portion define an angle 224 (FIG. 2) greater than about 100° and less than about 120°, such as greater than 100° and less than 120°, e.g., about 110°, or 110°. We believe this angle provides a comfortable grip position for many surgeons and avoids the above problems arising from scalpels having an approximate 90° angle between the axis 282 of the handle portion and the axis 283 of the body portion.

With reference to FIGS. 2-3, an embodiment of a surgical system 200, including an ultrasonic surgical handle assembly 201 in accordance with the present invention is illustrated. The surgical system 200 includes an ultrasonic generator 270 connected to an ultrasonic transducer 260 via cable 261, and an ultrasonic surgical handle assembly 201. It will be noted that, in some applications, the ultrasonic transducer 260 is referred to as a “hand piece assembly” because the surgical instrument of the surgical system 200 is configured such that a surgeon may grasp and manipulate the ultrasonic transducer 260 during various procedures and operations. A suitable generator is the GEN 300 sold by Ethicon Endo-Surgery, Inc. of Cincinnati, Ohio.

The ultrasonic surgical system 200 includes a multi-piece handle assembly 201 adapted to isolate the operator from the vibrations of the acoustic assembly contained within transducer 260. It is contemplated that the present surgical system 200 principally be grasped and manipulated by a trigger-like arrangement provided by a handle assembly 201 of the instrument, as will be described. While multi-piece handle assembly 201 is illustrated, the handle assembly 201 may comprise a single or unitary component. The proximal end of the ultrasonic surgical instrument handle assembly 201 receives and is fitted to the distal end of the ultrasonic transducer 260 by insertion of the transducer into the handle assembly 201. The ultrasonic surgical instrument handle assembly 201 may be attached to and removed from the ultrasonic transducer 260 as a unit. An elongated transmission assembly 204 of the ultrasonic surgical system 200 extends orthogonally from the instrument handle assembly 201. The transmission assembly 204 can be selectively rotated with respect to the handle assembly 201 as further described below. The handle assembly 201 may be constructed from a durable plastic, such as polycarbonate or a liquid crystal polymer. It is also contemplated that the handle assembly 201 may alternatively be made from a variety of suitable materials including other plastics, ceramics or metals.

The handle portion 202 (also referred to herein as “handle”) of the ultrasonic scalpel handle assembly 201 may be positioned with respect to the body portion 203 such that the axis 282 of the handle portion and the axis 283 of the body portion intersect at an angle 224 that is about 90° or greater than about 90°, e.g. about 90° to about 120°. The angle 224 can be chosen to provide an ergonomic grip design. In one embodiment, the angle 224 is about 110°, which can eliminate the need to hold the scalpel in a wrist-bent position during certain surgical procedures, thereby lowering operator fatigue. The construction of the ultrasonic scalpel shown in FIG. 2 enables a surgeon to operate the instrument when it is turned substantially in any direction, even if it is rotated at extreme angles (e.g., 120° along the axis of the scalpel body), while reducing the likelihood of errors in the application of energy during surgery.

The transmission assembly 204 (also referred to herein as “shaft”) may include an outer tubular member or outer sheath, an inner tubular actuating member, a waveguide and end-effector (blade 215, clamp arm 213 (also referred to herein as “clamp member”) and one or more clamp pads) as is known in the art. As will be described, the transmission assembly 204 may be joined together for rotation as a unit (together with ultrasonic transducer 260) relative to handle assembly 201. The waveguide, which is adapted to transmit ultrasonic energy from transducer 260 to blade 215 may be flexible, semi-flexible or rigid. The waveguide may also be configured to amplify the mechanical vibrations transmitted through the waveguide to the blade 215 as is well known in the art. The waveguide may further have features to control the gain of the longitudinal vibration along the waveguide and features to tune the waveguide to the resonant frequency of the system. In particular, the waveguide may have any suitable cross-sectional dimension. For example, the waveguide may have a substantially uniform cross-section or the waveguide may be tapered at various sections or may be tapered along its entire length. In one expression of the current embodiment, the waveguide diameter is about 0.113 inches nominal to minimize the amount of deflection at the blade 215 so that gapping in the proximal portion of the end-effector 280 is minimized.

The blade 215 may be integral with the waveguide and formed as a single unit. In an alternate expression of the current embodiment, blade 215 may be connected to the waveguide by a threaded connection, a welded joint, or other coupling mechanisms. The distal end of the blade 215 is disposed near an anti-node in order to tune the acoustic assembly to a preferred resonant frequency f₀ when the acoustic assembly is not loaded by tissue. When ultrasonic transducer 260 is energized, the distal end of blade 215 is configured to move longitudinally in the range of, for example, approximately 10 to 500 microns peak-to-peak, and preferably in the range of about 20 to about 200 microns at a predetermined vibrational frequency f₀ of, for example, 55,500 Hz. The above features of the waveguide and the transducer are known in the art.

Ultrasonic transducer 260, and an ultrasonic waveguide together provide an acoustic assembly of the present surgical system 200, with the acoustic assembly providing ultrasonic energy for surgical procedures when powered by generator 270. The acoustic assembly of surgical system 200 generally includes a first acoustic portion and a second acoustic portion. In the present embodiment, the first acoustic portion comprises the ultrasonically active portions of ultrasonic transducer 260, and the second acoustic portion comprises the ultrasonically active portions of transmission assembly 204. Further, in the present embodiment, the distal end of the first acoustic portion is operatively coupled to the proximal end of the second acoustic portion in any suitable manner, e.g., by, for example, a threaded connection.

A trigger 209 (also referred to herein as “squeeze trigger mechanism”, “trigger mechanism”, or “squeeze trigger”) opens the clamp member 213 to spread tissue and can be opened and closed using one or more fingers. As shown in FIG. 2, the trigger mechanism has a generally U-shaped groove. The U-shaped groove may be defined by a main surface 221 and an opposing surface 222 that are oriented substantially parallel to each other, and an adjoining surface 223 that is substantially perpendicular to and connects the main and opposing surfaces and is oriented opposite to an opening of the U-shaped groove. The trigger 209 of the ultrasonic scalpel shown in FIG. 2 is positioned toward the lower end of the handle portion 202 such that an activation mechanism 216 (e.g. a rocker switch for controlling the ultrasonic energy to the scalpel blade) can be located intermediate the trigger 209 and the body portion 203 and the transmission assembly 204 of the ultrasonic scalpel. This arrangement enables opening and closing of the clamp member 213 against the blade 215 with the ring finger 240 and little finger 250, while freeing up the index finger 220 and middle finger 230 to operate the actuation mechanism 216.

During operation, the clamp arm 213 is fully open relative to the blade 215 when the trigger 209 is in its most distal position. Ring finger 240 and little finger 250 may be placed within the U-shaped groove of the trigger 209 that is formed by surfaces 221, 222, 223 to actuate trigger 209 through its arcuate travel, which is designated by arrow 210. Movement of trigger 209 toward body portion 203 translates shaft 204 proximally, thereby pivoting clamp arm 213 toward blade 215. When the trigger 209 reaches its full proximal travel, the clamp arm 213 is in its fully closed position relative to the blade 215. In order to reverse the trigger 209 along its travel 210, fingers 250 and 260 push trigger 209 distally to open the end-effector 280. The clamp arm 213 is generally not biased open so moving the clamp arm 213 requires application of force to surface 222 of the trigger 209. The trigger-like action provided by trigger 209 and cooperating handle assembly 201 facilitates convenient and efficient manipulation and positioning of the instrument, and operation of the clamp arm 213 at the distal portion of the instrument whereby tissue is efficiently urged against the blade 215.

The squeeze trigger 209 can have an optional locking mechanism (not shown), wherein the trigger can be locked in a closed position or partially closed position to maintain an operator controlled, preset level of pressure between the clamp member 213 and the blade 215. This locking mechanism may also include a release button 217 elsewhere on the device, such as near the bottom of the body portion as shown in FIG. 2, or at another location on the handle assembly 201 or the handle portion 202. The locking mechanism may also be activated by squeezing the trigger completely; in some embodiments, the locking mechanism may be disengaged by squeezing the trigger again. Other locking mechanisms, such as described in WO 2006/042210, are known in the art may be used in combination with the scalpel of the present invention.

A shaft turning mechanism 208 (e.g., rotation knob) can be located at the periphery of the body portion 203 where the transmission assembly 204 protrudes from the body portion 203. The ultrasonic transducer 260, as well as the transmission assembly 204, is rotatable, as a unit, by suitable manipulation of shaft turning mechanism 208, relative to handle assembly 201 of the instrument. The interior of handle assembly 201 is dimensioned to accommodate such relative rotation of the ultrasonic transducer 260. This can be accomplished by providing slip ring conductors on the ultrasound transducer mounting boss within the handle assembly 201 that interfaces with ring contacts on the ultrasonic transducer 260 as described in WO 2006/042210. The activation mechanism 216 (e.g., rocker switch) is connected to posts in electrical communication with the slip ring conductors using wires or equivalent means.

In one expression of the current embodiment, the distal end of transducer 260 threadedly attaches to the proximal end of the waveguide which is located within the transmission assembly 204. The distal end of transducer 260 also interfaces with activation mechanism 216 to provide the surgeon with finger-activated controls on surgical handle assembly 201. The activation mechanism 216 is in electrical communication with the transducer 260 in a manner that permits rotation of the transducer 260 and transmission assembly 204 as discussed above.

One expression of the current embodiment allows the activation mechanism 216 to be configured in such a way to provide an ergonomic grip and operation for the surgeon. Activation mechanism 216 comprises a rocker switch positioned above the trigger 209 on the handle portion 202 of the handle assembly 201. Referring to FIG. 2, an example of the handle assembly 201 is shown graphically illustrating a surgeon's finger placement on handle assembly 201. The handle assembly 201 may be grasped by placement of the ring finger 240 and little finger 250 on trigger 209. (However, using the ring and little finger to activate trigger 209 is exemplary only. Surgeons may opt to activate trigger 209 with the middle finger 230 and ring finger 240, thereby making the index finger 220 available to activate the activation mechanism 216.) This finger placement allows the activation mechanism 216 to be controlled by the index finger 220 and/or middle finger 230. As an alternative to a rocker switch, the activation mechanism 216 may comprise a pair of pushbuttons that can be arranged above the trigger 209 on the handle body 202, in place of the illustrated rocker switch.

An optional electrocautery attachment 218a may be located on the top of the device or elsewhere on the handle, for example as with 218b, at the base of the handle. The electrocautery attachment allows electrocautery energy to pass through the device to the end effector 280 so the device can be used as a monopolar or possibly a bipolar cautery device. The electrocautery energy may be activated, e.g., by a mode selection switch 219, which may be located as shown in FIG. 2, or elsewhere on the handle assembly 201 or in a foot activated pedal (not shown). As an alternative to the separate electrocautery attachment 218, the electrocautery energy can be provided through the ultrasonic transducer attachment 260, which would provide energy for both electrocautery and ultrasonic operation. The device as shown in FIG. 2 is thus capable of applying both ultrasound or electrocautery energy to allow for dissection, cutting, and achieving hemostasis. However, ultrasonic scalpels according to the present invention may include only one form of energy for dissection, cutting, and/or achieving hemostasis, such as an ultrasonic-only scalpel.

The activation mechanism 216 may be utilized for controlling the degree or amount of energy delivered to the moveable blade. The activation mechanism 216 functions as an activation switch for switching on and off the electrocautery or ultrasonic energy to the blade 215 of the device or a toggle switch that controls the level of energy delivered to the ultrasonic scalpel at either the low, high, or other standard settings that may be defined, e.g., by manufacturer or the user. The ultrasonic scalpel may include a mode selection switch 219 that permits selecting between electrocautery or ultrasonic modes of operation. When the ultrasonic mode of operation is selected, the activation mechanism 216 may toggle between a low and high level of applied ultrasonic energy. Alternatively, or in addition to the rocker switch, a footpedal may be used to control the level of applied ultrasonic energy. When the electrocautery mode of operation is selected, the rocker switch may serve as an activation switch to turn on and off the applied electrocautery energy or it may toggle between low and high levels of applied electrocautery energy (e.g. where one setting corresponds to cutting and the other corresponds to coagulating functions). Alternatively, or in addition to the activation mechanism 216, a footpedal may be used to control the level of applied electrocautery energy. Other energy settings are possible depending on the requirements of the user. In one embodiment, a small indicator bump may be placed on either side of the rocker switch 216 to enable the user to press the correct button by feel. The rocker switch 216 may be positioned below the trigger 409 (or elsewhere on body 201) as an alternative to what is shown in FIG. 2.

FIG. 3 shows a cut out view of the ultrasonic scalpel depicted in FIG. 2 showing an exemplary arrangement of the trigger and mechanism for translating the reciprocal movement of the trigger into opening and closing of the clamp member 213 against the blade 215. It should be noted that FIG. 3 illustrates the handle assembly 301 while the ultrasound transducer 260 (See FIG. 2) is removed from the cavity 380. FIG. 3 shows an exemplary interaction between a trigger 309 and a collar 310 for actuating the opening and closing of the clamp member of the ultrasonic scalpel via means 320 comprising elongated slots and guide pins. Other mechanisms for opening and closing the clamp member against the blade are known by those having ordinary skill in the art. For instance, WO 2006/042210 A2 describes a mechanism utilizing a rotatable trigger and drive yoke for opening and closing a clamp arm against a blade. It should be appreciated that the design of various trigger mechanisms is within the capabilities of those having ordinary skill in the art. It will be appreciated that FIG. 3 illustrates only conceptually the cut out view of the ultrasonic scalpel, including only the details that are needed to show the arrangement of the trigger and mechanism for translating the reciprocal movement of the trigger into opening and closing the clamp member 213.

An ultrasonic scalpel as illustrated in FIG. 4 includes a body portion 401 (also referenced herein as a “body”), a shaft 404, a clamp member 403 and a blade 405, one of which is stationary (e.g., blade 405), and the other movable (e.g., blade 403). A trigger 409 opens the clamp member 403 to spread tissue and a substantially rectangular blade closure button 407 closes the clamp member 403. Two buttons, 413 and 414 (also referred to as “Max” and “Min”, respectively) control the degree or amount of energy delivered to the moveable blade. For instance, in an ultrasonic scalpel, the two buttons may control the amount of ultrasonic energy delivered to a blade of the ultrasonic scalpel. Buttons 413, 414 may alternatively actuate (or de-actuate) other features and functions used in ultrasonic scalpels, e.g., as known in the art. The shaft 404 may be rotated independent of the body of the instrument. The construction of the ultrasonic scalpel shown in FIG. 4 enables a surgeon to operate the instrument in any orientation, even if the body of the instrument is rotated 120° along the axis of the scalpel body.

As shown in FIG. 4, the body 401 of the ultrasonic scalpel may be barrel-shaped (or other substantially tubular-shaped) with a trigger 409 located adjacent the max/min actuation buttons 413, 414 for opening and closing the clamp member 403. A shaft turning mechanism 408 may be coupled to the body 401 and shaft 404 at the periphery of the body 401 where the shaft 404 protrudes from the body 401. A blade closure button 407 may be located on the body 401 opposite the trigger 409 in such a manner that it can be controlled with the user's hand irrespective of the position in which the scalpel is held. The ultrasonic scalpel may further comprise a stationary blade 405 opposite the clamp member 403. The ultrasonic scalpel may be adapted for electrocautery operation, whereby the electrocautery energy is controlled by a footswitch. A switching mechanism may be used to switch between electrocautery and ultrasonic operation whereby electrocautery or ultrasonic energy would be delivered to the stationary blade at a level that can be controlled by separate activator buttons 413 and 414. The level of applied energy can also be controlled using a rocker switch or other similar actuation (or variable actuation) mechanism that differentiates between two distinct power levels (not shown in FIG. 4). For instance, as an alternative to the rocker switch, a single button may be provided which has two settings that correspond to energy levels of separate activator buttons 413 and 414.

An ultrasonic scalpel as illustrated in FIG. 5 includes a body portion 501, a shaft 504, and an end-effector (not shown) comprising a clamp member and a blade. A trigger 509 may open the clamp member to spread tissue, and a blade closure mechanism 507 may close the moveable blade. A grip portion 502 may be provided on the same side of the body 501 as the trigger 509. In other embodiments, a grip portion 102 may be on a different side of the body 501. The grip portion 502 shown in FIG. 5 has an inclined grip positioned along the length of the body portion. A shaft turning mechanism 508 may be coupled to the body 501 and shaft 504 at the periphery of the body 501 where the shaft 504 protrudes from the body 501.

Two buttons 513 and 515 (also referred to as “Max” and “Min”, respectively) may control the degree or amount of energy delivered to the moveable blade. For instance, in an ultrasonic scalpel, the two buttons may control the amount of ultrasonic energy delivered to a blade of the ultrasonic scalpel. The construction of the ultrasonic scalpel shown in FIG. 5 may enable a surgeon to operate the instrument in any direction, even if it is rotated 120° along the axis of the scalpel body.

An ultrasonic scalpel as illustrated in FIG. 6 includes a body portion 601, a shaft 604, and an end-effector (not shown) comprising a clamp member and a blade. A trigger 609 may open the clamp member to spread tissue, and a blade closure button 607 may close the moveable blade. A grip portion 202 is provided on the same side of the body as the trigger 609 (or on a different side, not shown). The grip portion shown in FIG. 6 has an inclined grip positioned along the length of the body portion. A shaft turning mechanism 608 can be coupled to the body 601 and shaft 604 at the periphery of the body 201 where the transmission assembly 204 protrudes from the body 601. Two buttons, 613 and 615 (also referred to as “Max” and “Min”, respectively) may control the degree or amount of energy delivered to the moveable blade. For instance, in an ultrasonic scalpel, the two buttons may control the amount of ultrasonic energy delivered to a blade of the ultrasonic scalpel. The construction of the ultrasonic scalpel shown in FIG. 6 may enable a surgeon to operate the instrument in any orientation, even if it is rotated 120° along the axis of the scalpel body.

An ultrasonic scalpel as illustrated in FIG. 7 includes a body portion 701, a shaft 704, and an end-effector (not shown) comprising a clamp member and a blade. A trigger 709 may open the clamp member to spread tissue, and a blade closure button 707 may close the moveable blade. A grip portion 702 is provided on the same side of the body as the trigger 709. FIG. 7 illustrates an ultrasonic scalpel similar to that shown in FIG. 6, but with a grip section that is substantially aligned with (i.e., not inclined or at least less inclined from) the axis of the body portion. A shaft turning mechanism 708 can be located at the periphery of the body 701 where the shaft 704 protrudes from the body 701. Two buttons, 713 and 715 (also referred to as “Max” and “Min”, respectively) may control the degree or amount of energy delivered to the moveable blade. For instance, in an ultrasonic scalpel, the two buttons may control the amount of ultrasonic energy delivered to a blade of the ultrasonic scalpel. The construction of the ultrasonic scalpel shown in FIG. 7 may enable a surgeon to operate the instrument in any direction, even if it is rotated 120° along the axis of the scalpel body.

Other embodiments and uses of the invention will be apparent to those skilled in the art from consideration of the specification and practice of the invention disclosed herein. Numerous variations, changes, and substitutions will occur to those skilled in the art without departing from the scope of the invention. For instance, it should be appreciated that instead of (or in addition to) the buttons, triggers, and other actuators mentioned herein, e.g., buttons and trigger, other actuator devices may be used, such as switches, contacts, touch-sensitive actuators, and other mechanical or ultrasonic actuators. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A directed energy surgical instrument comprising:
a handle assembly comprising a body portion and a handle portion, the body portion and handle portion arranged in a pistol-shaped configuration;
an elongate transmission assembly attached to the body portion of the handle assembly and extending distally therefrom, the transmission assembly comprising a tubular sheath member attached at its proximal end to the body portion, an actuating member disposed within the tubular sheath member, and an end-effector assembly attached to the actuating member adjacent the distal end of the tubular sheath member, the end effector assembly comprising a blade member and a clamp member that is pivotable relative to the blade member, wherein the actuating member is configured for transmitting energy from the handle assembly to the end effector assembly;
a trigger mounted to the handle portion, the trigger being configured so that a portion of the trigger extends distally from a distal surface of the handle portion and being operatively connected to the actuating member so that motion of the trigger causes the clamp member to pivot relative to the blade member; and
an activation mechanism adapted to control the transmission of energy to the end effector assembly, wherein the activation mechanism is located on the distal surface of the handle portion intermediate the trigger and the body portion.

2. The directed energy surgical instrument of claim 1, wherein the trigger comprises a locking mechanism that holds the trigger in a closed or partially closed position to maintain an operator controlled, preset level of pressure between the clamp member and the blade member.

3. The directed energy surgical instrument of claim 2, wherein the locking mechanism comprises a release mechanism.

4. The directed energy surgical instrument of claim 3, wherein the release mechanism is activated by further depressing the trigger.

5. The directed energy surgical instrument of claim 3, wherein the release mechanism is activated by a button on the handle portion of the handle assembly.

6. The directed energy surgical instrument of claim 1 further comprising:
means for generating directed energy and transmitting said directed energy to the end effector assembly.

7. The directed energy surgical instrument of claim 6 wherein:
the directed energy is ultrasonic energy; and
the means for generating and transmitting includes an ultrasonic transducer.

8. The directed energy surgical instrument of claim 6 wherein:
the directed energy is electrocautery energy; and
the blade member is adapted for contact with and application of electrocautery energy to tissue.

9. The directed energy surgical instrument of claim 6, wherein the activation mechanism comprises a rocker switch in communication with the means for generating and transmitting, the rocker switch being configured for selectively controlling a level of directed energy to be transmitted to the end-effector assembly.

10. The directed energy surgical instrument of claim 1, wherein the handle portion and body portion of the handle assembly define an angle, the angle being greater than about 100° and less than about 120°.

11. The directed energy surgical instrument of claim 10, wherein the angle is about 110°.

12. The directed energy surgical instrument of claim 1, wherein the handle assembly can be gripped such that the index finger and middle finger contact the activation mechanism while the ring finger and little finger contact the trigger.

13. A directed energy surgical instrument comprising:
a handle assembly comprising a body portion and a handle portion, the body portion and handle portion arranged in a pistol-shaped configuration;
an elongate transmission assembly attached to the body portion of the handle assembly and extending distally therefrom, the transmission assembly comprising a tubular sheath member attached at its proximal end to the body portion, an actuating member disposed within the tubular sheath member, and an end-effector assembly attached to the actuating member adjacent the distal end of the tubular sheath member, the end effector assembly comprising a blade member and a clamp member that is pivotable relative to the blade member, wherein the actuating member is configured for transmitting energy from the handle assembly to the end effector assembly;
a trigger mounted to the handle portion, the trigger being configured so that a portion of the trigger extends distally from a distal surface of the handle portion and being operatively connected to the actuating member so that motion of the trigger causes the clamp member to pivot relative to the blade member; and
an activation means for controlling the transmission of energy to the end effector assembly, wherein the activation means is located on the distal surface of the handle portion intermediate the trigger and the body portion.

14. The directed energy surgical instrument of claim 13, wherein the trigger comprises a locking mechanism that holds the trigger in a closed or partially closed position to maintain an operator controlled, preset level of pressure between the clamp member and the blade member.

15. The directed energy surgical instrument of claim 14, wherein the locking mechanism comprises a release mechanism.

16. The directed energy surgical instrument of claim 15, wherein the release mechanism is activated by further depressing the trigger.

17. The directed energy surgical instrument of claim 15, wherein the release mechanism is activated by a button on the handle portion of the handle assembly.

18. The directed energy surgical instrument of claim 13 further comprising:
means for generating directed energy and transmitting said directed energy to the end effector assembly.

19. The directed energy surgical instrument of claim 18 wherein:
the directed energy is ultrasonic energy; and
the means for generating and transmitting includes an ultrasonic transducer.

20. The directed energy surgical instrument of claim 18 wherein:
the directed energy is electrocautery energy; and
the blade member is adapted for contact with and application of electrocautery energy to tissue.

21. The directed energy surgical instrument of claim 18, wherein the activation means comprises a rocker switch in communication with the means for generating and transmitting, the rocker switch being configured for selectively controlling a level of directed energy to be transmitted to the end-effector assembly.

22. The directed energy surgical instrument of claim 13, wherein the handle portion and body portion of the handle assembly define an angle, the angle being greater than about 100° and less than about 120°.

23. The directed energy surgical instrument of claim 22, wherein the angle is about 110°.

24. The directed energy surgical instrument of claim 13, wherein the handle assembly can be gripped such that the index finger and middle finger contact the activation means while the ring finger and little finger contact the trigger.
